(19) United States
(12) Patent Application Publication
(10) Pub. No.: US 2025/0009541 A1
(43) Pub. Date: Jan. 9, 2025

(54) WEARABLE MEDICAL DEVICE TO RELIEVE THE SYMPTOMS OF HAEMORRHOIDS AND METHOD OF USING THE SAME

(71) Applicant: Kishore Kiran, Kerala (IN)

(72) Inventor: Kishore Kiran, Kerala (IN)

(21) Appl. No.: 18/708,491

(22) PCT Filed: Oct. 27, 2022
(86) PCT No.: PCT/IN2022/000006

§ 371 Date: May 8, 2024

(30) Foreign Application Priority Data
Nov. 9, 2021 (IN) 202141051339

(51) Int. Cl. A61F 5/00

(52) U.S. Cl. CPC A61F 5/0093

(57) ABSTRACT

Disclosed herein is a peculiarly designed dome shaped (101) medical device (100) made of suitable flexible material that can be inserted into the anal canal (105), snugly fitted in between the buttocks (103) and worn with or without a string (108) with attachment (109) to hold it from falling off as shown in FIG. 2. The present invention is used to relieve the patient of various symptoms of piles or haemorrhoids like pain due to strangulation, burning sensation and also to prevent thrombosis instantly by pushing in the prolapsing haemorrhoid (106).

### BACKGROUND

### Field of the Invention

The present invention relates to a medical device used for relieving the symptoms of haemorrhoids or piles almost instantly. More particularly, it relates to a wearable medical device used in the anal canal to relieve the symptoms caused by prolapsing haemorrhoids or piles.

### Description of the Related Art

Haemorrhoids or piles are varicose veins of the anal canal. They are abnormally dilated anal vascular cushions. Haemorrhoids causes discomfort in the form of burning sensation, itching in the anal canal, bleeding per rectum, pain near the anal canal etc. In order to relieve the symptoms of haemorrhoids, it is important to understand why the symptoms of piles occur.

A person with normal anal vascular cushions has no symptoms and this vascular cushion helps in the normal defecation.

There are two reasons for haemorrhoids firstly genetic factors. Secondly a patient is more likely to develop piles over the years if he/she strains at stools for a prolonged period of time.

When a patient strains at stools, blood is forced into the venous plexus in the lower part of the anal canal. This venous congestion causes dilatation of the anal venous plexus. This process if repeated over a prolonged period of time results in haemorrhoids or piles. This means that a patient may have piles many years before he/she develop severe symptoms. They are actually varicose veins of the anal canal. Piles can be thought of or understood as a “bag of dilated veins” of the anal canal.

One of the most common symptoms of haemorrhoids is bleeding from the anal canal. If a patient with piles strains at stools so much that the dilated veins in the piles burst open, it can cause painless bleeding.

Venous congestion in the bag of veins can cause burning sensation in the anal canal. Microscopic bleeding from the haemorrhoidal veins causes histamine release which may cause itching in the anal canal.

After a few months or years the bag of dilated veins may become so big that it comes out during defecation and goes back inside after it. This is the reason for the feeling of mass per rectum which a patient feels during defection. Later on, the patient has to push the mass inside manually to reduce it.

The real reason for inventing the anal device is the identification of the fact that all of the symptoms of haemorrhoids are caused by the prolapsing of the haemorrhoidal mass out of the anal canal and the fact that reducing the prolapsed pile mass causes instant relief from all the symptoms due to haemorrhoids.

The real reason for symptoms of haemorrhoid is not inflammation as opposed to the general belief. But the inflammation of the haemorrhoidal veins is an effect of the mechanical obstruction of the blood to go back from the haemorrhoidal veins into the body due to straining at stools.

The key to finding a perfect cure for any disease is to find out the exact cause and correcting it. In the present case, the cause for the symptoms of piles is the prolapsing of the haemorrhoidal mass/venous plexus.

The inventor of present invention found that reduction of the pile mass causes instant symptomatic relief in all the patients of hemorrhoids.

Over the years there have been a wide variety of efforts made to relieve the discomfort and pain caused by haemorrhoids. Laxatives are widely used to treat symptoms of piles successfully by avoiding constipation thus reducing the prolapsing of pile mass.

There are a wide variety of ointments and other herbal preparations available in the market which are used for the same. But all of them are directed at reducing the inflammation of the haemorrhoidal veins. Other than that cushion seats, seat cushion rings, donut shaped ring cushion pillows are now available in the market. But none of these can give immediate or sustained relief as these measures do not address the actual cause of the symptoms of piles which is prolapsing haemorrhoidal mass/venous plexus.

### SUMMARY

A primary objective of the present invention is to obviate the above disadvantages by providing an absolutely novel anal device that can be worn by the patient. This dome shaped device made of silicone fits into the anal canal and relieves the patient of the various symptoms of haemorrhoids or piles instantly by pushing in the prolapsing haemorrhoid or reducing the prolapsing haemorrhoids. This device helps by relieving the kinking of the haemorrhoidal veins, promote venous return from the haemorrhoids, relieves symptoms of haemorrhoid like burning sensation, itching, perianal discomfort, pain due to strangulation and prevents thrombosis.

The medicines, laxatives, herbal preparations which are to be consumed may have a wide variety of side effects. But the present invention is completely devoid of any side effects but is much more effective than all the above measures in the symptomatic treatment of haemorrhoids or piles.

Though the present invention is not a cure for haemorrhoids, it can definitely reduce the symptoms of haemorrhoids till a corrective surgical procedure is carried out. Use of this device is similar to the use of compression socks or stockings for relieving the symptoms of varicose veins of the legs as both reduce the stretch on the vein walls. The present invention reduces the prolapsed pile mass by pushing it in. Hence, it does not have any therapeutic effect, prophylactic use and has no diagnostic value nor is part of a diagnostic device.

The device of the present invention is capable of being mass produced, marketed and sold at cheap price. This will bring immediate relief to numerous patients suffering from piles.

The present invention is a peculiarly designed dome shaped medical device made of silicone or other suitable material(s) that can be inserted into the anal canal and worn with or without a string to hold it in place while being mobile like walking, running or while being immobile by lying down or sitting down.

These objectives of the present invention will become more evident from the following detailed description when taken in conjunction with the accompanying drawings.

### BRIEF DESCRIPTION OF THE DRAWINGS

This invention will be more apparent from the ensuing description when read in conjunction with the accompanying drawings and wherein:

FIG. 1 shows the human anatomy of a patient suffering from haemorrhoids and the present invention; and

FIG. 2 represents as to how the medical device of the present invention is to be used.

### DETAILED WORKING OF THE INVENTION WITH REFERENCE TO ACCOMPANYING DRAWINGS

The present invention discloses a device and method for using the same for relieving the symptoms of piles or haemorrhoids.

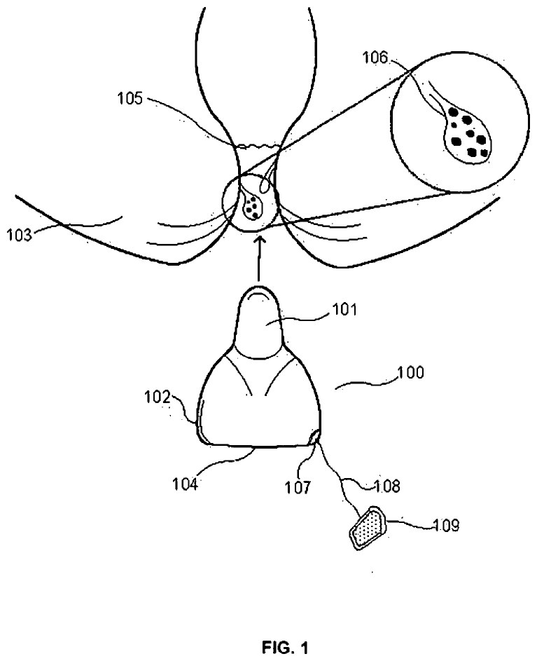

Referring to FIG. 1, The present invention discloses anatomy of buttocks 103 of a human suffering from haemorrhoids 106 and a medical device 100 made of silicon or any other suitable flexible material having a dome shape portion 101 at the top that can be inserted into the anal canal 105, side portions 102, bottom portion 104, a string inserting portion 107, a string to hang the device 108 and the portion that can be attached/anchored to the clothes 109.

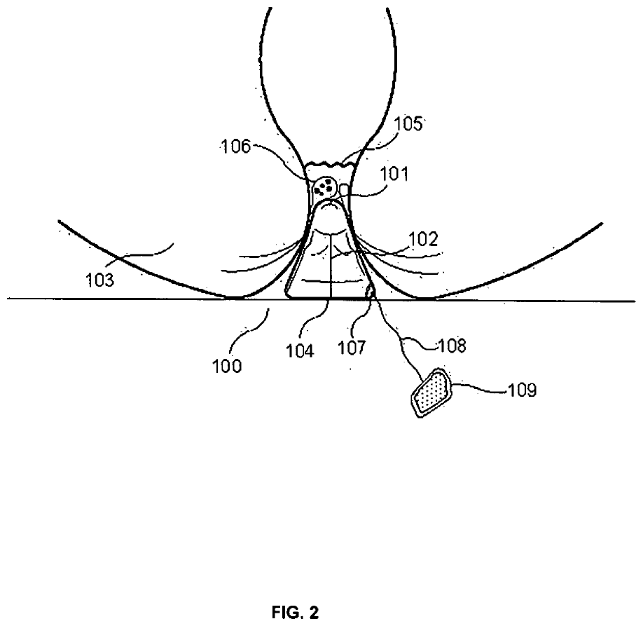

FIG. 2 shows as to how the medical device 100 is used. The dome shaped portion 101 is inserted into the anal canal 105 and side portion 102 snugly fits in between the buttocks 103. The dome shaped portion 101 pushes in the prolapsing haemorrhoids 106 and bottom portion 104 comes into touch with the surface where the patient sits and provides the necessary upward pressure and also helps the device to stay in place. The dome shaped part 101 of medical device 100 which fits into the anal canal intimately helps to reduce the haemorrhoidal mass and this reduction of haemorrhoidal mass help to relieve the kinking of the haemorrhoidal veins and promotes venous return thus relieving the pain and burning sensation. The string attached to the device vide 107 can be attached vide 109 to clothes for preventing it from falling off and creating an embarrassment. Any other means for holding the device in place can also be used.

Whereas, by employing device of the present invention symptoms of haemorrhoids or piles like burning sensation in the anal canal, itching, pain near the anal canal are considerably reduced by pushing in the prolapsed haemorrhoid vein or mass.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope of the invention as claimed.

1. A medical device capable of being inserted into the anal canal of a patient for relieving the haemorrhoidal symptoms and preventing complications comprising:
a) a dome shaped top portion (101) capable of being inserted into the anal canal (105), side portions (102) capable of fitting snugly in between the buttocks (103), bottom portion (104) which comes into contact with surface while sitting, the groove for holding the string (107), string (108) and anchor for attaching to clothes (109).

2. The medical device as claimed in claim 1, is made of silicone or any other suitable material.

3. The medical device as claimed in claim 1, is capable of being worn by the patient with or without a string to hold it in place while being mobile and immobile.

4. A method of using a medical device for relieving the haemorrhoidal symptoms, wherein the method comprising:
a) inserting a dome shaped top portion of the device into the anal canal; and
b) attaching the string or other holding means to the device and to the patient's clothes.
